(19) United States
(12) Patent Application Publication
(10) Pub. No.: US 2011/0054574 A1
(43) Pub. Date: Mar. 3, 2011

(54) Ultraviolet sterilizer for surgery

(76) Inventor: Perry Felix, Houston, TX (US)

(21) Appl. No.: 12/807,022

(22) Filed: Aug. 26, 2010

Related U.S. Application Data
(60) Provisional application No. 61/275,135, filed on Aug. 26, 2009.

(51) Int. Cl. A61N 5/06; A61L 2/10

(52) U.S. Cl. 607/92; 607/94; 250/493.1

(57) ABSTRACT

An ultraviolet sterilizer for use during surgery is mounted in a base cabinet. The UV light source can be a laser, or an LED. An optical frequency multiplier can be used that outputs UV of less than 280 nm, or greater than 320 nm, to avoid burning the patient. A visible LED aiming light directs the UV light toward the surgery. A crosshair image can be projected to position the light.

One lamp has a housing, a cavity, a handle, and an ocular plate to pass the UV and the aiming light. An articulated arm allows selective positioning of the lamp. Another lamp has a stylus, a handle, and a tip small enough for easy insertion into a small incision for arthroscopy. A fiber optic cable connects the UV and the aiming light to the lamp. Lenses or filters can be used with the fiber optic cable.

An electronic power supply and a CPU connect to the UV and the aiming light sources. A keyboard inputs commands to the CPU. A sensor provides feedback.

Another UV sterilizer is mounted on a ceiling of the operating room. A lamp has a housing with a cavity. Either a curved or a flat substrate is mounted in the cavity. Solid state UV elements are arrayed on the substrate, along with visible LEDs for aiming. Either a curved or a flat mirror is disposed behind the substrate. An ocular plate passes the UV and the aiming light, and protects the elements from damage. The ocular plate is a diffuser, a filter, or a fresnel lens.

### CROSS-REFERENCE TO RELATED APPLICATIONS

Reference is hereby made to provisional patent application titled, “Method and Apparatus For Sanitizing and Sterilization In Surgery Using an Ultraviolet Coherent Light Source;” filed by Perry Dean Felix, of Houston, Tex., on Aug. 26, 2009, Ser. No. 61/275,135. The prior application is expressly incorporated herein by reference.

### STATEMENT REGARDING FEDERALLY SPONSORED RESEARCH OR DEVELOPMENT

Not Applicable

### BACKGROUND OF THE INVENTION

This invention relates to the field of ultraviolet sterilization, and more particularly to sanitizing and sterilizing for medical and surgical applications by using an ultraviolet coherent light source or a high intensity focused ultraviolet light source.

Surgery has become commonplace worldwide in medical practice to maintain the health and save the lives of patients. Surgery to repair, remove, or replace damaged organs, blood vessels, and tissues. Surgery to remove cancerous growths. Surgery to enhance appearance cosmetically. This includes surgery of all kinds including, but not limited to, general surgery, dental and oral surgery, open heart surgery, laparoscopic surgery, arthroscopic surgery, and veterinary surgery. In the course of these surgical procedures, open incisions and exposed tissues render the patient vulnerable to infection by bacteria and viruses. Maintenance of a sterile operating environment has proven to be difficult. Oral and intravenous antibiotics and topical antiseptics are not always effective in preventing infections.

Introducing topical antiseptics during surgery can interrupt the surgeon's concentration. Introducing topical antiseptics during laparoscopic and arthroscopic surgery is difficult through the tiny incisions. Covering the entire exposed tissue area with an antiseptic during general surgery is not always possible. Many people are allergic to certain intravenous antibiotics, sometimes with life-threatening consequences. Topical antiseptics sterilize only the applied surfaces, and do not penetrate the tissues.

Accordingly, there is a need to provide an ultraviolet sterilizer for use during surgery that can be administered while the surgeon operates, as well as during a pause in the procedure, as desired.

There is a further need to provide an ultraviolet sterilizer of the type described for use during surgery and that can be administered during laparoscopic and arthroscopic surgery through tiny incisions.

There is a yet further need to provide an ultraviolet sterilizer of the type described for use during surgery and that will consistently cover the entire exposed tissue area, and penetrate through layers of tissue to destroy bacteria beneath the surface.

There is a still further need to provide an ultraviolet sterilizer of the type described for use during surgery and that will reliably destroy every class of bacterial and viral germs, without harming the patient.

There is another need to provide an ultraviolet sterilizer of the type described for use during surgery and that is completely non-allergenic to every patient.

There is yet another need to provide an ultraviolet sterilizer of the type described for use during surgery and that can be manufactured cost-effectively while maintaining standards of high quality.

### BRIEF SUMMARY OF THE INVENTION

In accordance with the present invention, there is provided an ultraviolet sterilizer for use during surgery 20. The ultraviolet sterilizer 20 comprises a base 22, and an ultraviolet (UV) light source 24 received within the base 22. The UV light source 24 can be of any type, for example a UV vertical cavity surface emitting laser, a UV light emitting diode (LED), a UV vertical light emitting diode, an edge emitting laser, a fiber laser, or a lamp. UV light wavelengths for the application may range from 90 nm to 400 nm. UV light in the band of wavelengths between 280 nm and 320 nm is known to burn living tissues. Certain kinds of UV light sources will require an optical frequency multiplier that outputs light in the ultraviolet range, and having a wavelength of less than 280 nm, or greater than 320 nm, to avoid burning the patient.

An aiming light source 26 is received within the base 22 to selectively direct the UV light toward the surgery. The aiming light source 26 can be integral with the UV light source 24, or separate. Their outputs are combined optically into one beam. A crosshair image can be projected to position the light on the incision.

A lamp 28 has a housing 30 with a cavity 32 and at least one handle 34 adapted for manual grasping. An ocular plate 36 will pass the ultraviolet light and the aiming light outward. An articulated arm 44 between the base 22 and the lamp 28 supports and allows selective positioning of the lamp 28.

An alternative lamp 128 comprises a stylus 130 with a handle 132 adapted for manual grasping. The stylus 130 has a tip 134 smaller in diameter than the handle 132, for easy insertion into any small incision or enclosed space. Examples include laparoscopic incisions, arthroscopic incisions, and oral cavities.

A fiber optic cable 38 extends from a proximal end 40 connected to the UV light source 24 and the aiming light source 26, to a distal end 42 connected to the lamp. Various lenses or filters can be placed at either end of the fiber optic cable 38.

An electronic power supply 46 is received within the base 22, to power the ultraviolet light source 24 and the aiming light source 26.

A computer having a central processor unit 48 is received within the base, to control the ultraviolet light source 24 and the aiming light source 26. A keyboard 50 inputs commands to the central processor unit 48. A sensing means measures parameters of the ultraviolet light, and provides feedback to the central processor unit 48.

Another embodiment of the ultraviolet sterilizer 320 is mounted on a ceiling of the operating room. A lamp 352 has a housing 354 with a cavity 356 and at least one handle 358 adapted for manual grasping. Either a curved substrate 360 or a flat substrate 378 is received within the housing cavity 356. The substrate serves as a foundation for mounting electronic components on one side, and a wiring harness 362 on the opposite side. The curved substrate 360 helps focus the light.

A plurality of solid state UV light emitting elements 364 are arrayed on the substrate 360 or 378. The UV elements 364 are preferably vertical cavity surface emitting lasers. A plurality of solid state visible light emitting elements 366, preferably LEDs, are also arrayed on the substrate 360 or 378 for aiming. The UV elements 364 and the visible LEDs 366 can be arrayed in any desired pattern on the substrate 360 or 378. The UV elements 364 and the LEDs 366 are electrically connected to a power supplying means (not shown) and to a controlling means (not shown) by a wire cable 363.

Either a curved mirror 368 or a flat mirror 386 is received within the housing cavity 356 and is disposed behind the substrate 360 or 378 to reflect light outward. An ocular plate 370 is mounted on the housing 354 to pass the ultraviolet light and the aiming light outward. The ocular plate 370 can be a diffuser, a filter, a fresnel lens, or any element that will pass and process the light. The ocular plate 370 further serves to protect the UV elements 364 and the LEDs 366 from moisture, damage, and soiling.

A base 322 is attached to the ceiling. An articulated arm 344 is interposed between the base 322 and the lamp 352 for supporting and selectively positioning the second lamp 352, and for directing the light toward the surgery.

### BRIEF DESCRIPTION OF THE SEVERAL VIEWS OF THE DRAWING

A more complete understanding of the present invention may be obtained from consideration of the following description in conjunction with the drawing, in which:

FIG. 1 is a perspective, partial sectional view of an ultraviolet sterilizer constructed in accordance with the invention.

FIG. 2 is a perspective, partial sectional view of another ultraviolet sterilizer constructed in accordance with the invention.

FIG. 3 is a perspective, detail view of a further embodiment of the ultraviolet sterilizer of FIG. 2.

FIG. 4 is a perspective, partial sectional view of yet another ultraviolet sterilizer constructed in accordance with the invention.

FIG. 5 is a perspective, exploded detail view of the ultraviolet sterilizer of FIG. 4.

FIG. 6 is a perspective, exploded detail view of an additional embodiment of the ultraviolet sterilizer of FIG. 4.

FIG. 7 is a perspective, exploded view of still another ultraviolet sterilizer constructed in accordance with the invention.

FIG. 8 is a perspective, exploded detail view of an additional embodiment of the ultraviolet sterilizer of FIG. 7.

### DETAILED DESCRIPTION OF THE INVENTION

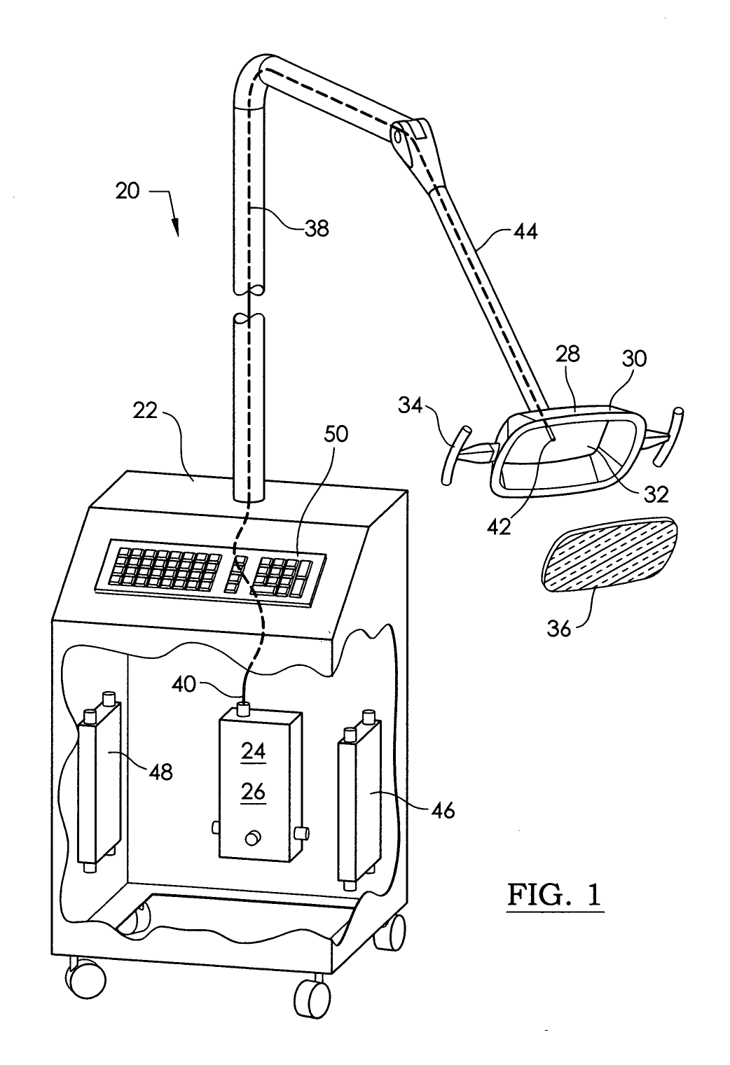

Referring now to the drawing, and especially to FIGS. 1-4 thereof, an ultraviolet sterilizer for use during surgery is shown at 20. The ultraviolet sterilizer 20 comprises a base 22, and an ultraviolet light source 24 received within the base 22 for supplying ultraviolet light. The ultraviolet (UV) light source 24 can be of any type, for example an ultraviolet vertical cavity surface emitting laser, an ultraviolet vertical light emitting diode (LED), an edge emitting laser, and a fiber laser. A solid state source is preferred, but any ultraviolet light source 24 can be employed, as for instance a mercury vapor UV lamp. UV light in the band of wavelengths between 280 nm and 320 nm is known to cause sunburn and like damage to living tissues. Certain kinds of ultraviolet light sources 24 will require an optical frequency multiplier that outputs light in the ultraviolet range, and having a wavelength of less than 280 nm, or greater than 320 nm, as to avoid burning the patient. For example, a fiber laser emitting a light wavelength of 1064 nm is frequency tripled to yield an output having a wavelength of 355 nm, which is above the damaging band. Another laser emitting a light wavelength of 1064 nm is frequency doubled twice using optics and crystals to quadruple the frequency to yield an output having a wavelength of 266 nm, which is below the damaging band.

An aiming light source 26, is received within the base 22. As the ultraviolet light is invisible, a visible aiming light is necessary to selectively direct the ultraviolet light toward the surgery. A solid state aiming light source 26 such as a visible LED, is preferred. However, any visible source can be used, such as a halogen light. In FIG. 1, the aiming light source 26 is shown as integral with the ultraviolet light source 24. Their outputs are combined optically into one beam. The aiming light may include further aids, including a means (not shown) for the projection of geometric patterns. A contrasting image such as a crosshair or target rings can be projected, to help the surgeon position the light properly on the incision. Projection of images is well known by those skilled in the art.

A lamp is shown at 28, and is in optical communication with the ultraviolet light source 24, and the aiming light source 26. The lamp 28 has a housing 30 with a cavity 32 and at least one handle 34 adapted for manual grasping. An ocular plate 36 is mounted on the housing 30 to pass the ultraviolet light and the aiming light outward toward the surgery. The ocular plate 36 can be a diffuser, a filter, a fresnel lens, or any element that will pass light outward, and process the light as desired. An articulated arm 44 is interposed between the base 22 and the lamp 28 for supporting and selectively positioning the lamp 28 and for directing the aiming light toward the surgery.

A fiber optic cable 38 extends from a proximal end 40 connected to the ultraviolet light source 24 and the aiming light source 26, through the articulated arm 44, to a distal end 42 connected to the lamp 28. The fiber optic cable 38 conducts the combined beam of the ultraviolet light and the aiming light to the lamp 28. The distal end 42 of the fiber optic cable 38 is polished to allow light to pass. Various lenses or filters (not shown) can be introduced into the light path at either end of the fiber optic cable 38 to manage the light.

Power supplying means 46 is received within the base 22, for supplying electrical power to the ultraviolet light source 24 and the aiming light source 26. The power supplying means 46 is an electronic power supply 46 receiving power from an external source (not shown). The electronic power supply 46 is electrically connected to the ultraviolet light source 24 and the aiming light source 26. The connections are not shown in FIG. 1, but are well known by those skilled in the art.

Controlling means is received within the base, for controlling the ultraviolet light source and the aiming light source. The controlling means is a computer having a central processor unit (CPU) 48 connected to the ultraviolet light source 24 and the aiming light source 26. An inputting means, such as a keyboard 50, is provided for inputting commands to the central processor unit 48. A sensing means (not shown) is provided for sensing parameters of the ultraviolet light, such as intensity and frequency. The sensing means provides feedback to the central processor unit 48. The feedback data is analyzed and used to regulate the ultraviolet light source 24.

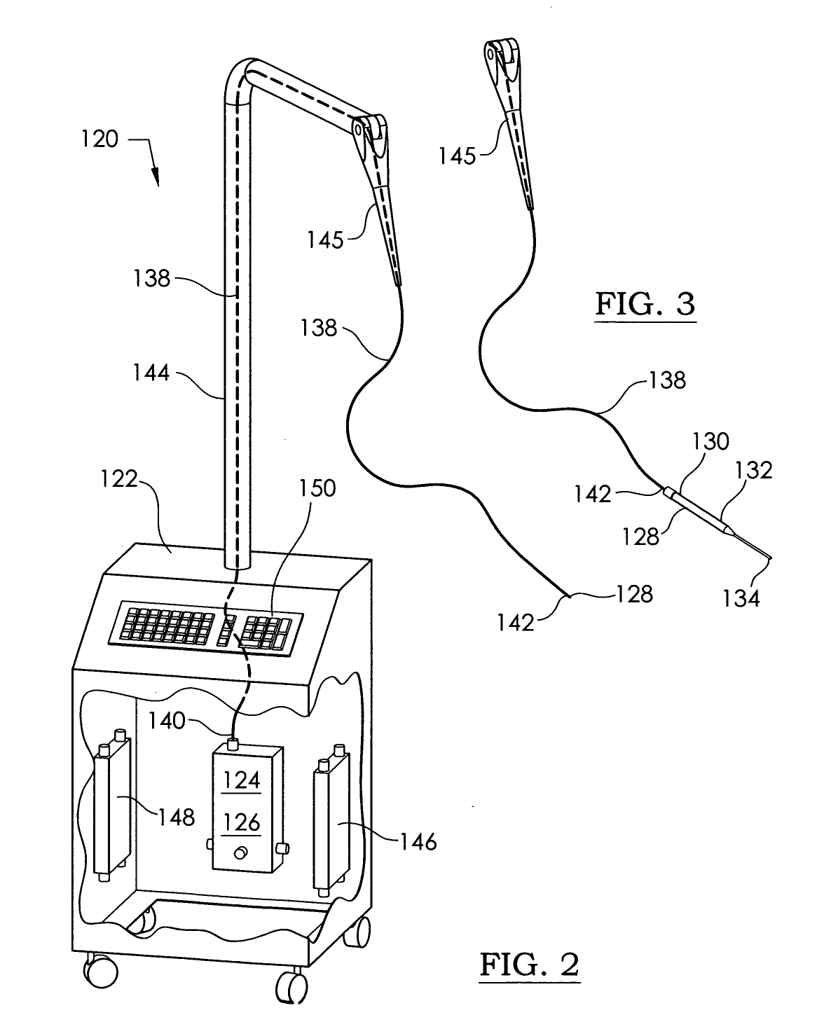

Turning now to FIGS. 2 and 3, another ultraviolet sterilizer for use during surgery is shown at 120. The ultraviolet sterilizer 120 is similar to ultraviolet sterilizer 20 described above, in that it comprises a base 122, and an ultraviolet light source 124 received within the base 122 for supplying ultraviolet light. As before, a solid state source is preferred, but any ultraviolet light source 124 can be employed. An optical frequency multiplier may be used to adjust the output to be within the proper band.

An aiming light source 126, is received within the base 122. A solid state aiming light source 126 such as a visible LED, is preferred. In FIG. 2, the aiming light source 126 is shown as integral with the ultraviolet light source 124. Their outputs are combined optically into one beam. The aiming light may include a projected crosshair or target rings for positioning.

A lamp is shown at 128, and is in optical communication with the ultraviolet light source 124, and the aiming light source 126. The ultraviolet sterilizer 120 differs from ultraviolet sterilizer 20 described above, in that the lamp 128 further comprises a stylus 130 with a handle 132 adapted for manual grasping. The stylus 130 has a tip 134 extending from the handle 132. The tip 134 is smaller in diameter than the handle 132, so that the tip 134 can be easily inserted into any small incision or enclosed space. Examples include laparoscopic incisions, arthroscopic incisions, and oral cavities.

An arm 144 is rotatably mounted upon the base 122 and may optionally include an articulated member 145. A fiber optic cable 138 extends from a proximal end 140 connected to the ultraviolet light source 124 and the aiming light source 126, through the arm 144, to a distal end 142 connected to the lamp 128. The fiber optic cable 138 conducts the combined beam of the ultraviolet light and the aiming light to the lamp 128. The distal end 142 of the fiber optic cable 138 is polished to allow light to pass. Optionally, the lamp 128 can comprise only the polished distal end 142 of the fiber optic cable 138, without the stylus 130.

A power supplying means, specifically an electronic power supply 146, is received within the base 122, for supplying electrical power to the ultraviolet light source 124 and the aiming light source 126. The electronic power supply 146 is electrically connected to the ultraviolet light source 124 and the aiming light source 126.

A controlling means, specifically a computer having a central processor unit 148, is received within the base 122, and is connected to the ultraviolet light source 124 and the aiming light source 126. An inputting means, such as a keyboard 150, is provided for inputting commands to the central processor unit 148. A sensing means (not shown) is provided for sensing parameters of the ultraviolet light, and supplying feedback to the central processor unit 148.

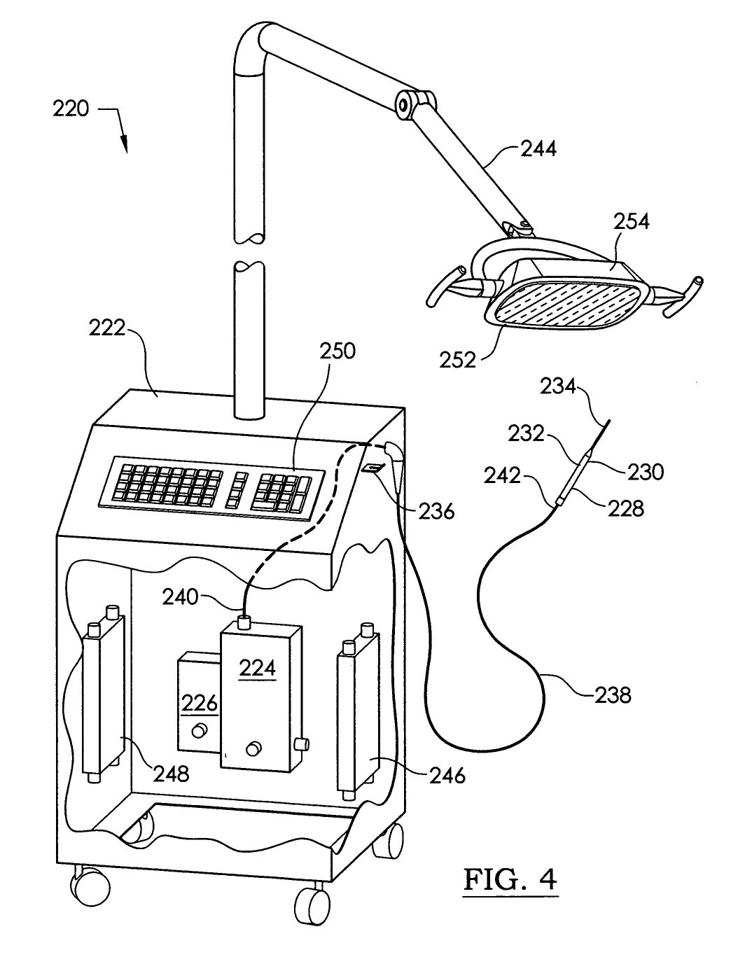

Referring now to FIGS. 4, 5, and 6, yet another embodiment of the ultraviolet sterilizer for use during surgery is shown at 220. The ultraviolet sterilizer 220 is similar to ultraviolet sterilizer 20 described above, in that it comprises a base 222, and an ultraviolet light source 224 received within the base 222 for supplying ultraviolet light. As before, a solid state source is preferred, but any ultraviolet light source 224 can be employed. An optical frequency multiplier may be used to adjust the output to be within the proper band.

An aiming light source 226, is received within the base 222. A solid state aiming light source 226 such as a visible LED, is preferred. In FIG. 4, the aiming light source 226 is shown as a separate unit mounted adjacent the ultraviolet light source 224. Their outputs are combined optically into one beam. The aiming light may include a projected crosshair or target rings for positioning.

A first lamp is shown at 228, and is in optical communication with the ultraviolet light source 224, and the aiming light source 226. The ultraviolet sterilizer 220 differs from ultraviolet sterilizer 20 described above, in that the first lamp 228 further comprises a stylus 230 with a handle 232 adapted for manual grasping. The stylus 230 has a tip 234 extending from the handle 232. The tip 234 is smaller in diameter than the handle 232, so that the tip 234 can be easily inserted into any small incision or enclosed space. Examples include laparoscopic incisions, arthroscopic incisions, and oral cavities.

A fiber optic cable 238 extends from a proximal end 240 connected to the ultraviolet light source 224 and the aiming light source 226, to a distal end 242 connected to the first lamp 228. The fiber optic cable 238 conducts the combined beam of the ultraviolet light and the aiming light to the first lamp 228. The flexible fiber optic cable 238 allows the surgeon to comfortably manipulate the lamp in any direction. The distal end 242 of the fiber optic cable 238 is polished to allow light to pass. Optionally, the first lamp 228 can comprise only the polished distal end 242 of the fiber optic cable 238, without the stylus 230. A hook 236 holds the stylus 230 when it is not in use.

A power supplying means, specifically an electronic power supply 246, is received within the base 222, for supplying electrical power to the ultraviolet light source 224 and the aiming light source 226. The electronic power supply 246 is electrically connected to the ultraviolet light source 224 and the aiming light source 226.

A computer having a central processor unit 248 is received within the base 222, and is connected to the ultraviolet light source 224 and the aiming light source 226. An inputting means, such as a keyboard 250, is provided for inputting commands to the central processor unit 248. A sensing means (not shown) is provided for sensing parameters of the ultraviolet light. The sensor supplies feedback to the central processor unit 248.

A second lamp is shown at 252 in FIGS. 4 and 5. The second lamp 252 has a housing 254 with a cavity 256 and at least one handle 258 adapted for manual grasping. A curved substrate 260 is received within the housing cavity 256. The curved substrate 260 serves as a foundation for mounting electronic components on one side. The curved substrate 260 is curved to help focus and concentrate the light. The components are connected on the opposite side to one another or to an external wiring harness 262.

A second ultraviolet light source comprises a plurality of solid state ultraviolet light emitting elements 264 arrayed on the substrate 260. The solid state ultraviolet light emitting elements 264 are preferably vertical cavity surface emitting lasers. Other solid state devices can be used, for example, UV vertical light emitting diodes.

A second aiming light source comprises a plurality of solid state visible light emitting elements 266 arrayed on the substrate 260. The solid state visible light emitting elements 266 are preferably light emitting diodes. The ultraviolet light emitting elements 264 and the visible light emitting elements 266 can be arrayed in any desired pattern on the substrate 260. For example, the elements can alternate with one another across length and width of the substrate area. In another example, the visible light emitting elements 266 can be clustered in a central area, surrounded by the ultraviolet light emitting elements 264.

The ultraviolet light emitting elements 264 and the visible light emitting elements 266 are electrically connected to the electronic power supply 246 and to the central processor unit 248 by a wire cable 263.

A curved mirror 268 is received within the housing cavity 256 and is disposed behind the curved substrate 260. The curved mirror 268 helps to reflect light outward.

An ocular plate 270 is mounted on the housing 254 to pass the ultraviolet light and the aiming light outward toward the surgery. The ocular plate 270 can be a diffuser, a filter, a fresnel lens, or any element that will pass light outward, and process the light as desired. The ocular plate 270 further serves to protect the ultraviolet light emitting elements 264 and the visible light emitting elements 266 from moisture, damage, and soiling.

An articulated arm 244 is interposed between the base 222 and the second lamp 252 for supporting and selectively positioning the second lamp 252, and for directing the light toward the surgery.

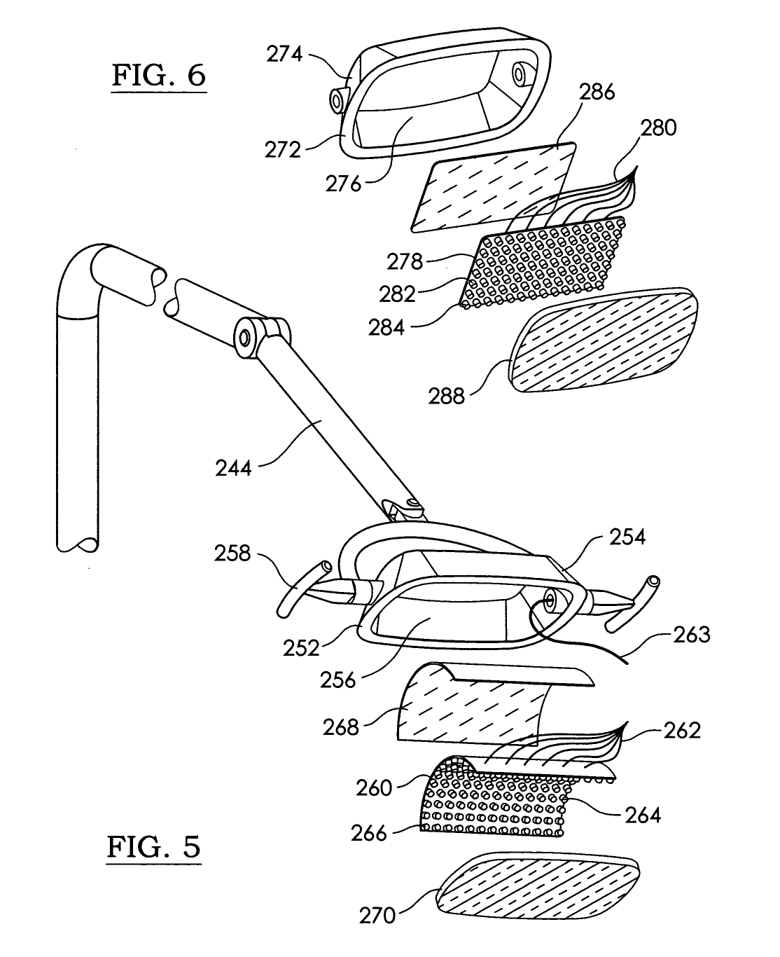

A alternative second lamp 272 is shown in FIG. 6. The second lamp 272 has a housing 274 with a cavity 276. A flat substrate 278 is received within the housing cavity 276. The flat substrate 278 takes up less space in the lamp housing 274, and relies upon the ocular plate for focusing the light. The components are connected on to an external wiring harness 280.

A plurality of solid state ultraviolet light emitting elements 282 are arrayed on the flat substrate 278. A plurality of solid state visible light emitting elements 284 are arrayed on the flat substrate 278. As before, the elements 282 and 284 can be arrayed in any desired pattern on the substrate 278.

A flat mirror 286 is received within the housing cavity 276 and is disposed behind the flat substrate 278. The flat mirror 286 helps to reflect light outward.

An ocular plate 288 is mounted on the housing 274 to pass the ultraviolet light and the aiming light outward toward the surgery.

An optional filter (not shown) can be interposed into the optical path at any point. The filter is configured to block the passage of ultraviolet light having a wavelength between 280 nm and 320 nm, so as to avoid burning the patient. The filter is a redundant safety measure to protect the patient. The filter can be included in any embodiment of the invention described herein.

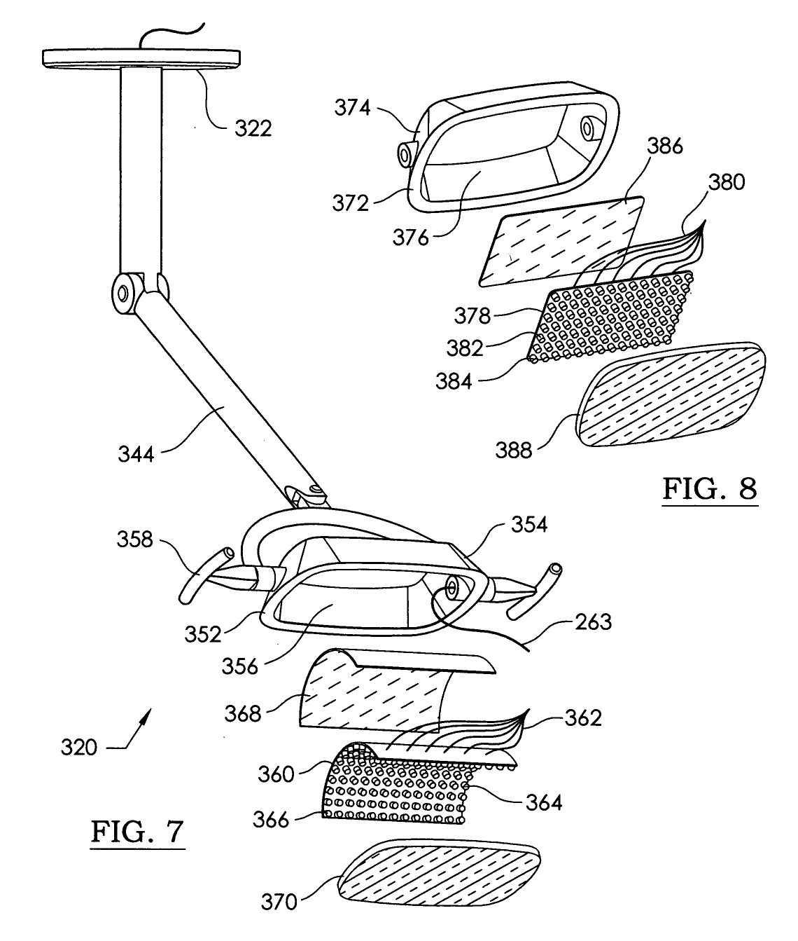

Referring now to FIGS. 7 and 8, still another embodiment of the ultraviolet sterilizer for use during surgery is shown at 320. The ultraviolet sterilizer 320 is similar to the second lamp 252 of ultraviolet sterilizer 220 described above, in that it comprises a lamp 352 with a housing 354 having a cavity 356 and at least one handle 358 adapted for manual grasping. A curved substrate 360 is received within the housing cavity 356. The curved substrate 360 serves as a foundation for mounting electronic components on one side. The curved substrate 360 is curved to help focus and concentrate the light. The components are connected on the opposite side to one another or to an external wiring harness 362.

An ultraviolet light source comprises a plurality of solid state ultraviolet light emitting elements 364 arrayed on the substrate 360. The solid state ultraviolet light emitting elements 364 are preferably vertical cavity surface emitting lasers. Other solid state devices can be used, for example, UV vertical light emitting diodes.

An aiming light source comprises a plurality of solid state visible light emitting elements 366 arrayed on the substrate 360. The solid state visible light emitting elements 366 are preferably light emitting diodes. The ultraviolet light emitting elements 364 and the visible light emitting elements 366 can be arrayed in any desired pattern on the substrate 360. For example, the elements can alternate with one another across length and width of the substrate area. In another example, the visible light emitting elements 366 can be clustered in a central area, surrounded by the ultraviolet light emitting elements 364.

The ultraviolet light emitting elements 364 and the visible light emitting elements 366 are electrically connected to a power supplying means (not shown) and to a controlling means (not shown) by a wire cable 363. The controlling means can be only a switch, or can be a central processor unit.

A curved mirror 368 is received within the housing cavity 356 and is disposed behind the curved substrate 360. The curved mirror 368 helps to reflect light outward.

An ocular plate 370 is mounted on the housing 354 to pass the ultraviolet light and the aiming light outward toward the surgery. The ocular plate 370 can be a diffuser, a filter, a fresnel lens, or any element that will pass light outward, and process the light as desired. The ocular plate 370 further serves to protect the ultraviolet light emitting elements 364 and the visible light emitting elements 366 from moisture, damage, and soiling.

A base 322 is attached to a ceiling. An articulated arm 344 is interposed between the base 322 and the lamp 352 for supporting and selectively positioning the second lamp 352, and for directing the light toward the surgery.

A alternative lamp 372 is shown in FIG. 8. The lamp 372 has a housing 374 with a cavity 376. A flat substrate 378 is received within the housing cavity 376. The flat substrate 378 takes up less space in the lamp housing 374, and relies upon the ocular plate for focusing the light. The components are connected on to an external wiring harness 380.

A plurality of solid state ultraviolet light emitting elements 382 are arrayed on the flat substrate 378. A plurality of solid state visible light emitting elements 384 are arrayed on the flat substrate 378. As before, the elements 382 and 384 can be arrayed in any desired pattern on the substrate 378.

A flat mirror 386 is received within the housing cavity 376 and is disposed behind the flat substrate 378. The flat mirror 386 helps to reflect light outward.

An ocular plate 388 is mounted on the housing 374 to pass the ultraviolet light and the aiming light outward toward the surgery.

Using the ultraviolet sterilizer involves a sequence of events that typically will start with turning the power on, upon which the CPU will boot up, and then execute a system self test and calibrate the sensor. The surgeon will then position the ultraviolet sterilizer over the surgery or wound area, utilizing the visible light aiming. This is augmented with graphic or crosshair positioning aids. The UV system is triggered for a duration of 30 seconds or so. The layer of surgery is closed. The process is continued and repeated for each layer until the surgery is completed.

A method of sterilizing with ultraviolet light is also disclosed, for use during surgery, the method comprising the steps of supplying ultraviolet light with an ultraviolet light source, supplying visible aiming light with an aiming light source, providing a base, then providing a lamp, and communicating the lamp optically with the ultraviolet light source and the aiming light source. Next, directing the ultraviolet light toward the surgery with the aiming light, supplying electrical power to the ultraviolet light source and the aiming light source, and controlling the ultraviolet light source and the aiming light source, selectively.

Further steps comprise connecting a proximal end of a fiber optic cable to the ultraviolet light source and the aiming light source, connecting a distal end of the fiber optic cable to the lamp, and conducting the ultraviolet light and the aiming light to the lamp through the fiber optic cable. Next, receiving the ultraviolet light source and the aiming light source within the base, and selecting the ultraviolet light source from the group consisting of an ultraviolet vertical cavity surface emitting laser, an ultraviolet vertical light emitting diode, an edge emitting laser, and a fiber laser.

Further steps include multiplying the frequency of the ultraviolet light source optically, and outputting light in the ultraviolet range with a wavelength of less than 280 nm, thereby avoiding burning the patient.

Yet further steps comprise multiplying the frequency of the ultraviolet light source optically, and outputting light in the ultraviolet range with a wavelength of greater than 320 nm, thereby avoiding burning the patient.

Still further steps comprise interposing an articulated arm between the base and the lamp, and supporting and selectively positioning the lamp with the articulated arm, then providing the lamp with a housing having a cavity, mounting an ocular plate on the housing, and passing the ultraviolet light and the aiming light outward toward the surgery through the ocular plate.

Additional steps comprise providing a stylus as the lamp, extending a handle along the stylus, and adapting the handle for manual grasping, then projecting a tip from the handle, the tip being smaller in diameter than the handle, and adapting the tip for inserting into a laparoscopic incision, an arthroscopic incision, and an oral cavity.

Additional steps include controlling the ultraviolet light with a central processor unit, inputting commands to the central processor unit, then sensing parameters of the ultraviolet light, and providing feedback to the central processor unit.

Yet additional steps comprise interposing an articulated arm between the base and the lamp, and supporting and selectively positioning the lamp with the articulated arm, then providing the lamp with a housing having a cavity, and receiving a substrate within the housing cavity. Next, arraying a plurality of solid state ultraviolet light elements on the substrate as the ultraviolet light source, arraying a plurality of solid state visible light elements on the substrate as the aiming light source, then mounting an ocular plate on the housing, and passing the ultraviolet light and the aiming light outward toward the surgery through the ocular plate.

A still additional step comprises projecting a geometric pattern having a contrasting image along with the visible aiming light, for positioning the light on the surgery.

Numerous modifications and alternative embodiments of the invention will be apparent to those skilled in the art in view of the foregoing description. Accordingly, this description is to be construed as illustrative only and is for the purpose of teaching those skilled in the art the best mode of carrying out the invention. Details of the structure may be varied substantially without departing from the spirit of the invention and the exclusive use of all modifications that will come within the scope of the appended claims is reserved.

### PARTS LIST

### Sterilization for Surgery Using Ultraviolet Light

| PART | |
|---|---|
| NO. | DESCRIPTION |
| 20 | ultraviolet sterilizer |
| 22 | base |
| 24 | ultraviolet light source |
| 26 | aiming light source |
| 28 | lamp |
| 30 | lamp housing |
| 32 | lamp housing cavity |
| 34 | lamp housing handle |
| 36 | ocular plate |
| 38 | fiber optic cable |
| 40 | fiber optic cable proximal end |
| 42 | fiber optic cable distal end |
| 44 | articulated arm |
| 46 | electronic power supply |
| 48 | central processor unit |
| 50 | keyboard |
| 120 | ultraviolet sterilizer |
| 122 | base |
| 124 | ultraviolet light source |
| 126 | aiming light source |
| 128 | lamp |
| 130 | stylus |
| 132 | stylus handle |
| 134 | stylus tip |
| 138 | fiber optic cable |
| 140 | fiber optic cable proximal end |
| 142 | fiber optic cable distal end |
| 144 | arm |
| 145 | articulated member |
| 146 | electronic power supply |
| 148 | central processor unit |
| 150 | keyboard |
| 220 | ultraviolet sterilizer |
| 222 | base |
| 224 | ultraviolet light source |
| 226 | aiming light source |
| 228 | first lamp |
| 230 | stylus |
| 232 | stylus handle |
| 234 | stylus tip |
| 236 | hook |
| 238 | fiber optic cable |
| 240 | fiber optic cable proximal end |
| 242 | fiber optic cable distal end |
| 244 | articulated arm |
| 246 | electronic power supply |
| 248 | central processor unit |
| 250 | keyboard |
| 252 | second lamp |
| 254 | housing |
| 256 | cavity |
| 258 | handle |
| 260 | curved substrate |
| 262 | wiring harness |
| 263 | wire cable |
| 264 | UV light emitting elements |
| 266 | visible light emitting elements |
| 268 | curved mirror |
| 270 | ocular plate |
| 272 | alternative second lamp |
| 274 | housing |
| 276 | cavity |
| 278 | flat substrate |
| 280 | wiring harness |
| 282 | UV light emitting elements |
| 284 | visible light emitting elements |
| 286 | flat mirror |
| 288 | ocular plate |
| 320 | ultraviolet sterilizer |
| 322 | base |
| 344 | articulated arm |
| 352 | lamp |
| 354 | housing |
| 356 | cavity |
| 358 | handle |
| 360 | curved substrate |
| 362 | wiring harness |
| 363 | wire cable |
| 364 | UV light emitting elements |
| 366 | visible light emitting elements |
| 368 | curved mirror |
| 370 | ocular plate |
| 372 | alternative lamp |
| 374 | housing |
| 376 | cavity |
| 378 | flat substrate |
| 380 | wiring harness |
| 382 | UV light emitting elements |
| 384 | visible light emitting elements |
| 386 | flat mirror |
| 388 | ocular plate |

The embodiments of the invention in which an exclusive property or privilege is claimed are defined as follows:

1. An ultraviolet sterilizer, for use during surgery, the ultraviolet sterilizer comprising:
an ultraviolet light source for supplying ultraviolet light;
an aiming light source for supplying visible aiming light so as to selectively aim the ultraviolet light;
a base;
a lamp, the lamp being in optical communication with the ultraviolet light source and the aiming light source for directing the ultraviolet light and the aiming light toward the surgery, the lamp being supported by the base;
power supplying means for supplying electrical power to the ultraviolet light source and the aiming light source, the power supplying means being electrically connected to the ultraviolet light source and the aiming light source; and
controlling means for controlling the ultraviolet light source and the aiming light source, the controlling means being connected to the ultraviolet light source and the aiming light source.

2. The ultraviolet sterilizer of claim 1, wherein:
a fiber optic cable is included, the fiber optic cable extending from a proximal end connected to the ultraviolet light source and the aiming light source, to a distal end connected to the lamp, so as to conduct the ultraviolet light and the aiming light to the lamp;
the ultraviolet light source, the aiming light source, the power supplying means, and the controlling means are received within the base; and
the ultraviolet light source is selected from the group consisting of: an ultraviolet vertical cavity surface emitting laser, an ultraviolet vertical light emitting diode, an edge emitting laser, and a fiber laser.

3. The ultraviolet sterilizer of claim 2, wherein the ultraviolet light source further comprises an optical frequency multiplier that outputs light in the ultraviolet range, and having a wavelength of less than 280 nm, so as to avoid burning the patient.

4. The ultraviolet sterilizer of claim 2, wherein the ultraviolet light source further comprises an optical frequency multiplier that outputs light in the ultraviolet range, and having a wavelength of greater than 320 nm, so as to avoid burning the patient.

5. The ultraviolet sterilizer of claim 2, further comprising:
an articulated arm interposed between the base and the lamp for supporting and selectively positioning the lamp;
the lamp having a housing with a cavity and at least one handle adapted for manual grasping; and
an ocular plate mounted on the housing so as to pass the ultraviolet light and the aiming light outward toward the surgery.

6. The ultraviolet sterilizer of claim 2, wherein the lamp further comprises a stylus, the stylus having a handle adapted for manual grasping, and a tip extending from the handle, the tip being smaller in diameter than the handle, for insertion of the tip into a laparoscopic incision, an arthroscopic incision, and an oral cavity.

7. The ultraviolet sterilizer of claim 2, wherein the controlling means further comprises:
a central processor unit;
an inputting means for inputting commands to the central processor unit; and
a sensing means for sensing parameters of the ultraviolet light, the sensing means providing feedback to the central processor unit.

8. The ultraviolet sterilizer of claim 1, further comprising:
an articulated arm interposed between the base and the lamp for selectively positioning the lamp;
the lamp having a housing with a cavity and at least one handle adapted for manual grasping;
a substrate received within the housing cavity;
the ultraviolet light source having a plurality of solid state ultraviolet light emitting elements arrayed on the substrate;
the aiming light source having a plurality of solid state visible light emitting elements arrayed on the substrate; and
an ocular plate mounted on the housing so as to pass the ultraviolet light and the aiming light outward toward the surgery.

9. The ultraviolet sterilizer of claim 8, further comprising a mirror received within the housing cavity and disposed behind the substrate, to reflect light outward.

10. The ultraviolet sterilizer of claim 1, further comprising a filter to block the passage of ultraviolet light having a wavelength between 280 nm and 320 nm, so as to avoid burning the patient.

11. The ultraviolet sterilizer of claim 1, wherein the aiming light source further comprises projecting means for projecting a geometric pattern having a contrasting image, so as to position the light on the surgery.

12. An ultraviolet sterilizer, for use during surgery, the ultraviolet sterilizer comprising:
a base;
an ultraviolet light source received within the base for supplying ultraviolet light, the ultraviolet light source being selected from the group consisting of: an ultraviolet vertical cavity surface emitting laser, an ultraviolet vertical light emitting diode, an edge emitting laser, and a fiber laser;
a solid state aiming light source, received within the base, for supplying visible aiming light so as to selectively aim the ultraviolet light;
a lamp, the lamp being in optical communication with the ultraviolet light source and the aiming light source for directing the ultraviolet light and the aiming light toward the surgery, the lamp being supported by the base;
power supplying means, received within the base, for supplying electrical power to the ultraviolet light source and the aiming light source, the power supplying means being electrically connected to the ultraviolet light source and the aiming light source;
controlling means, received within the base, for controlling the ultraviolet light source and the aiming light source, the controlling means being connected to the ultraviolet light source and the aiming light source; and
a fiber optic cable extending from a proximal end connected to the ultraviolet light source and the aiming light source, to a distal end connected to the lamp, so as to conduct the ultraviolet light and the aiming light to the lamp.

13. The ultraviolet sterilizer of claim 12, wherein the controlling means further comprises:
a central processor unit;
an inputting means for inputting commands to the central processor unit; and
a sensing means for sensing parameters of the ultraviolet light, the sensing means providing feedback to the central processor unit.

14. The ultraviolet sterilizer of claim 12, further comprising:
an articulated arm interposed between the base and the lamp for supporting and selectively positioning the lamp;
the lamp having a housing with a cavity and at least one handle adapted for manual grasping; and
an ocular plate mounted on the housing so as to pass the ultraviolet light and the aiming light outward toward the surgery.

15. The ultraviolet sterilizer of claim 12, wherein the lamp further comprises a stylus, the stylus having a handle adapted for manual grasping, and a tip extending from the handle, the tip being smaller in diameter than the handle, for insertion of the tip into a laparoscopic incision, an arthroscopic incision, and an oral cavity.

16. The ultraviolet sterilizer of claim 12, wherein the ultraviolet light source further comprises an optical frequency multiplier that outputs light in the ultraviolet range, and having a wavelength of less than 280 nm, so as to avoid burning the patient.

17. The ultraviolet sterilizer of claim 12, wherein the ultraviolet light source further comprises an optical frequency multiplier that outputs light in the ultraviolet range, and having a wavelength of greater than 320 nm, so as to avoid burning the patient.

18. An ultraviolet sterilizer, for use during surgery, the ultraviolet sterilizer comprising:
a base;
a lamp having a housing with a cavity and at least one handle adapted for manual grasping, the lamp being supported by the base;
an articulated arm interposed between the base and the lamp for selectively directing the lamp toward the surgery;
a substrate received within the housing cavity;
an ultraviolet light source for supplying ultraviolet light, the ultraviolet light source having a plurality of solid state ultraviolet light emitting elements arrayed on the substrate;
an aiming light source for supplying visible aiming light so as to selectively aim the ultraviolet light, the aiming light source having a plurality of solid state visible light emitting elements arrayed on the substrate;
power supplying means for supplying electrical power to the ultraviolet light source and the aiming light source, the power supplying means being electrically connected to the ultraviolet light source and the aiming light source;
controlling means for controlling the ultraviolet light source and the aiming light source, the controlling means being connected to the ultraviolet light source and the aiming light source; and
an ocular plate mounted on the housing so as to pass the ultraviolet light and the aiming light outward toward the surgery.

19. The ultraviolet sterilizer of claim 18, further comprising a mirror received within the housing cavity and disposed behind the substrate, to reflect light outward.

20. A method of sterilizing with ultraviolet light, for use during surgery, the method comprising the steps of:
supplying ultraviolet light with an ultraviolet light source;
supplying visible aiming light with an aiming light source;
providing a base;
providing a lamp, and communicating the lamp optically with the ultraviolet light source and the aiming light source;
directing the ultraviolet light toward the surgery with the aiming light;
supplying electrical power to the ultraviolet light source and the aiming light source; and
controlling the ultraviolet light source and the aiming light source, selectively

21. The method of claim 20, further comprising the steps of:
connecting a proximal end of a fiber optic cable to the ultraviolet light source and the aiming light source;
connecting a distal end of the fiber optic cable to the lamp;
conducting the ultraviolet light and the aiming light to the lamp through the fiber optic cable;
receiving the ultraviolet light source and the aiming light source within the base; and
selecting the ultraviolet light source from the group consisting of: an ultraviolet vertical cavity surface emitting laser, an ultraviolet vertical light emitting diode, an edge emitting laser, and a fiber laser;

22. The method of claim 21, further comprising the steps of:
multiplying the frequency of the ultraviolet light source optically; and
outputting light in the ultraviolet range with a wavelength of less than 280 nm; thereby
avoiding burning the patient.

23. The method of claim 21, further comprising the steps of:
multiplying the frequency of the ultraviolet light source optically; and
outputting light in the ultraviolet range with a wavelength of greater than 320 nm; thereby
avoiding burning the patient.

24. The method of claim 21, further comprising the steps of:
Interposing an articulated arm between the base and the lamp, and supporting and selectively positioning the lamp with the articulated arm;
providing the lamp with a housing having a cavity; and
mounting an ocular plate on the housing, and passing the ultraviolet light and the aiming light outward toward the surgery through the ocular plate.

25. The method of claim 21, further comprising the steps of:
providing a stylus as the lamp;
extending a handle along the stylus, and adapting the handle for manual grasping;
projecting a tip from the handle, the tip being smaller in diameter than the handle; and
adapting the tip for inserting into a laparoscopic incision, an arthroscopic incision, and an oral cavity.

26. The method of claim 21, further comprising the steps of:
controlling the ultraviolet light with a central processor unit;
inputting commands to the central processor unit; and
sensing parameters of the ultraviolet light, and providing feedback to the central processor unit.

27. The method of claim 20, further comprising the steps of:
Interposing an articulated arm between the base and the lamp, and supporting and selectively positioning the lamp with the articulated arm;
providing the lamp with a housing having a cavity;
receiving a substrate within the housing cavity;
arraying a plurality of solid state ultraviolet light elements on the substrate as the ultraviolet light source;
arraying a plurality of solid state visible light elements on the substrate as the aiming light source; and
mounting an ocular plate on the housing, and passing the ultraviolet light and the aiming light outward toward the surgery through the ocular plate.

28. The method of claim 20, further comprising the step of projecting a geometric pattern having a contrasting image along with the visible aiming light, for positioning the light on the surgery.
